(19) United States
(12) Patent Application Publication
(10) Pub. No.: US 2025/0001099 A1
(43) Pub. Date: Jan. 2, 2025

(54) SYRINGE OUTER CYLINDER, SYRINGE, AND PREFILLED SYRINGE

(71) Applicant: TERUMO KABUSHIKI KAISHA, Tokyo (JP)

(72) Inventor: Junichi OGAWA, Kofu-shi (JP)

(73) Assignee: TERUMO KABUSHIKI KAISHA, Tokyo (JP)

(21) Appl. No.: 18/885,951

(22) Filed: Sep. 16, 2024

Related U.S. Application Data
(63) Continuation of application No. PCT/JP2022/045543, filed on Dec. 9, 2022.

(30) Foreign Application Priority Data
Mar. 18, 2022 (JP) 2022-044681

(51) Int. Cl. A61M 5/34; A61M 5/32

(52) U.S. Cl. CPC A61M 5/34; A61M 5/3202

(57) ABSTRACT

A syringe outer cylinder includes: an outer cylinder including: a nozzle part located at a distal end of the outer cylinder, and a locking cylindrical part covering the nozzle part; a cap attachable to and detachable from the outer cylinder; and an elastic sealing member for sealing a distal opening of the nozzle part, the elastic sealing member being housed in the cap.

### CROSS-REFERENCE TO RELATED APPLICATIONS

This is a bypass continuation of PCT Application No. PCT/JP2022/045543, filed on Dec. 9, 2022, which claims priority to Japanese Patent Application No. 2022-044681, filed on Mar. 18, 2022. The entire contents of these applications are incorporated herein by reference.

### BACKGROUND

The present disclosure relates to a syringe outer cylinder, a syringe and prefilled syringe using the same.

In PCT Publication No. WO2014/102987 (US Patent Publication No. 2015-190566A, European Patent Publication No. 2939649A; “Patent Literature 1”), the applicant of the present application has proposed a syringe outer cylinder that includes an outer cylinder body 60, a nozzle part 64 provided at a distal portion of the outer cylinder body 60 and having a distal opening part 63 at a distal end, a lock adapter 70 that covers the nozzle part 64, and a cap 80 that seals the distal opening part 63 of the nozzle part 64. Furthermore, the cap 80 includes a sealing member 87 that is housed in an inside cylindrical part 84 and seals the distal opening part 63 of the nozzle part 64 of the outer cylinder body 60.

Furthermore, a gap between an inner peripheral surface of an outside cylindrical part 83 of the cap 80 and an outer peripheral surface of the inside cylindrical part 84 of the cap 80 is formed in a size that allows entrance of a cylindrical body part 71 of the lock adapter 70, and the outer peripheral surface of the inside cylindrical part 84 is provided with a cap-side screw part 85 that can be screwed with an adapter-side screw part (cylindrical part-side screw part) 79 of the lock adapter 70. Furthermore, a connection part 89 of the cap 80 and a distal end of the cylindrical body part 71 of the lock adapter 70 are in contact with each other to prevent excessive tightening of the adapter-side screw part 79 and the cap-side screw part 85.

### SUMMARY

The syringe outer cylinder of Patent Literature 1 is effective for preventing excessive tightening of the adapter-side screw part and the cap-side screw part. However, to reliably seal a distal opening of the nozzle part of the outer cylinder body, it is necessary to press the sealing member with a certain degree of force. When the cap is attached to the outer cylinder at a level that does not cause excessive tightening, the sealing member is pressed and elastically deformed. Furthermore, the elastically deformed sealing member generates repulsive force to return to an original shape. Due to the repulsive force, the cap may be detached from the outer cylinder. In particular, the inventor of the present application has found that the cap is likely to be detached from the outer cylinder as an amount of squeezing the sealing member increases.

An object of certain embodiments of the present disclosure is to provide a syringe outer cylinder in which a cap is attached to a nozzle part of an outer cylinder, a sealing member is sufficiently compressed, a distal opening of a nozzle can be reliably sealed, and the cap is less likely to be detached from the outer cylinder even if an amount of squeezing the sealing member is large, and a syringe and prefilled syringe using the same.

According to one embodiment, a syringe outer cylinder includes an outer cylinder including a nozzle part provided on a distal end, and a locking cylindrical part covering the nozzle part, a cap attachable to and detachable from the outer cylinder, and an elastic sealing member for sealing a distal opening of the nozzle part, the elastic sealing member being housed in the cap. The cap is a cylindrical member, and includes a sealing-member housing part that houses the elastic sealing member, a closing part positioned on an upper portion of the sealing-member housing part, a nozzle-part housing part that extends downward from the sealing-member housing part and houses the nozzle part, a cap-side screw part formed on an outer surface of the nozzle-part housing part, and a gap portion formed, at least before being attached to the outer cylinder, between an upper portion of the sealing-member housing part and an upper surface peripheral portion of the elastic sealing member, or between an inner-side portion of the sealing-member housing part and an outer-side portion of the elastic sealing member. The locking cylindrical part of the outer cylinder includes, in an inner surface, a locking cylindrical part-side screw part that is configured to be screwed with the cap-side screw part. The elastic sealing member is compressed by an inner surface of the cap and a distal end of the nozzle part and elastically deformed when the cap is attached to the outer cylinder, and a swollen portion formed by the elastic deformation is positioned in the gap portion.

According to another embodiment, a syringe includes the syringe outer cylinder described above and a gasket slidably housed in the syringe outer cylinder.

According to another embodiment, a prefilled syringe includes the syringe outer cylinder having the cap attached to the outer cylinder, a gasket slidably housed in the syringe outer cylinder, and medical liquid housed in the syringe outer cylinder.

### BRIEF DESCRIPTION OF THE DRAWINGS

FIG. 1 is a front view of a syringe outer cylinder according to an embodiment of the present invention.

FIG. 2 is a cross-sectional view taken along the line A-A in FIG. 1.

FIG. 3 is an enlarged cross-sectional view of a distal portion of the syringe outer cylinder illustrated in FIG. 2.

FIG. 4 is an enlarged cross-sectional view of a distal portion of a syringe outer cylinder according to another embodiment of the present invention.

FIG. 5 is an enlarged front view of a cap used in the syringe outer cylinder illustrated in FIG. 1.

FIG. 6 is a plan view of the cap illustrated in FIG. 5.

FIG. 7 is a bottom view of the cap illustrated in FIG. 5.

FIG. 8 is a cross-sectional view taken along the line B-B in FIG. 5.

FIG. 9 is a cross-sectional view taken along the line C-C in FIG. 6.

FIG. 10 is a cross-sectional view taken along the line D-D in FIG. 6.

FIG. 11 is a front view of a sealing member used for the syringe outer cylinder according to the embodiment of the present invention.

FIG. 12 is a plan view of the sealing member illustrated in FIG. 11.

FIG. 13 is a bottom view of the sealing member illustrated in FIG. 11.

FIG. 14 is an enlarged cross-sectional explanatory view for describing a sealing member of the syringe outer cylinder according to the another embodiment of the present invention.

FIG. 15 is a front view of a prefilled syringe using the syringe outer cylinder according to the embodiment of the present invention.

FIG. 16 is a cross-sectional view taken along the line E-E in FIG. 15.

FIG. 17 is a plan view of the syringe outer cylinder according to the another embodiment of the present invention.

FIG. 18 is an enlarged cross-sectional view of a distal portion of the syringe outer cylinder, taken along the line F-F in FIG. 17.

FIG. 19 is an enlarged cross-sectional view of the distal portion of the syringe outer cylinder, taken along the line G-G in FIG. 17.

FIG. 20 is a cross-sectional view taken along the line H-H in FIG. 18.

FIG. 21 is a front view of a sealing member used for the syringe outer cylinder illustrated in FIG. 18.

FIG. 22 is a plan view of the sealing member illustrated in FIG. 21.

FIG. 23 is a bottom view of the sealing member illustrated in FIG. 21.

FIG. 24. is an enlarged cross-sectional explanatory view for describing a state of a sealing member in a cap before being attached to the syringe outer cylinder in FIG. 17.

FIG. 25 is an enlarged bottom view of a cap used for the syringe outer cylinder illustrated in FIG. 18.

FIG. 26 is a cross-sectional view taken along the line J-J in FIG. 25.

FIG. 27 is a cross-sectional view taken along the line K-K in FIG. 25.

FIG. 28 is a front view of a sealing member of another example, the sealing member being used for the syringe outer cylinder of the present invention.

FIG. 29 is a plan view of the sealing member illustrated in FIG. 28.

FIG. 30 is a bottom view of the sealing member illustrated in FIG. 28.

FIG. 31 is an enlarged cross-sectional explanatory view for describing a state in which the sealing member illustrated in FIG. 28 is attached to the cap illustrated in FIG. 25.

FIG. 32 is an enlarged cross-sectional explanatory view for describing a state in which the sealing member illustrated in FIG. 28 is attached to the cap illustrated in FIG. 25 and further attached to an outer cylinder.

FIG. 33 is a front view of a sealing member of another example, the sealing member being used for the syringe outer cylinder of the present invention.

FIG. 34 is a plan view of the sealing member illustrated in FIG. 33.

FIG. 35 is a bottom view of the sealing member illustrated in FIG. 33.

FIG. 36 is a front view of a sealing member of another example, the sealing member being used for the syringe outer cylinder of the present invention.

FIG. 37 is a plan view of the sealing member illustrated in FIG. 36.

FIG. 38 is a bottom view of the sealing member illustrated in FIG. 36.

FIG. 39 is an enlarged cross-sectional explanatory view for describing a state in which the sealing member illustrated in FIG. 36 is attached to the cap illustrated in FIG. 25.

FIG. 40 is an enlarged cross-sectional explanatory view for describing a state in which the sealing member illustrated in FIG. 36 is attached to the cap illustrated in FIG. 25 and further attached to an outer cylinder.

FIG. 41 is a front view of a sealing member of another example, the sealing member being used for the syringe outer cylinder according to the embodiment of the present invention.

FIG. 42 is a plan view of the sealing member illustrated in FIG. 41.

FIG. 43 is a bottom view of the sealing member illustrated in FIG. 41.

FIG. 44 is a front view of a sealing member of another example, the sealing member being used for the syringe outer cylinder according to the embodiment of the present invention.

FIG. 45 is a plan view of the sealing member illustrated in FIG. 44.

FIG. 46 is a bottom view of the sealing member illustrated in FIG. 44.

FIG. 47 is a longitudinal sectional view of a prefilled syringe of another embodiment, the prefilled syringe using the syringe outer cylinder of the present invention.

### DETAILED DESCRIPTION

A syringe outer cylinder of the present disclosure and a syringe and prefilled syringe using the same will be described by using embodiments illustrated in the drawings.

A syringe outer cylinder 1, 1a according to an embodiment of the present disclosure includes an outer cylinder 2, 2a including a nozzle part 22 provided at a distal end thereof and a locking cylindrical part (4, 25) covering the nozzle part 22, a cap 3 detachably attached to the outer cylinder 2, and an elastic sealing member 6 housed in the cap 3 and sealing a distal opening of the nozzle part 22.

The cap 3 is a cylindrical member, and includes a sealing-member housing part 31 that houses the elastic sealing member 6, a closing part 32 positioned at an upper portion of the sealing-member housing part 31, a nozzle-part housing part 33 that extends downward from the sealing-member housing part 31 and houses the nozzle part 22, a cap-side screw part 33a formed on an outer surface of the nozzle-part housing part 33, and a gap portion 13, 14 formed, at least before being attached to an outer cylinder, between an upper portion of the sealing-member housing part 31 and an upper surface peripheral portion of the elastic sealing member 6 or between an inner-side portion of the sealing-member housing part 31 and an outer-side portion of the elastic sealing member 6. On an inner surface thereof, a locking cylindrical part 4 of the outer cylinder 2 includes a locking cylindrical part-side screw part 41a that can be screwed with the cap-side screw part 33a. When the cap is attached to the outer cylinder, the elastic sealing member 6 is compressed by an inner surface of the cap 3 and a distal end of the nozzle part 22 and elastically deformed, and a swollen portion 62, 64 formed with the elastic deformation is positioned in the gap portion 13, 14.

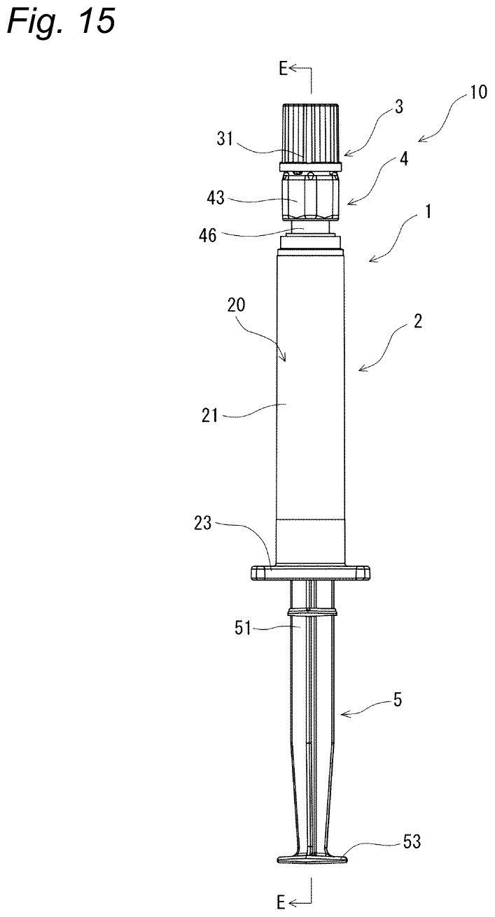

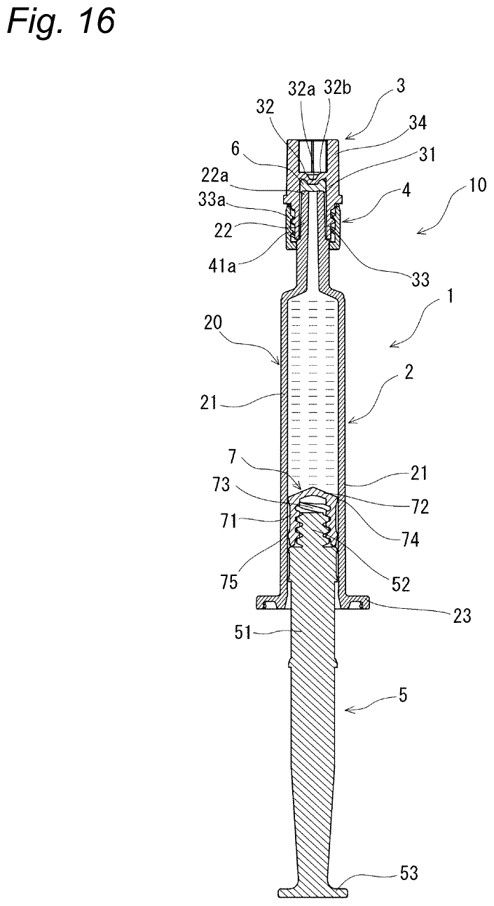

In addition, as illustrated in FIGS. 15, 16, and 47, the syringe of the present invention includes the syringe outer cylinder 1, 1a and a gasket 7 slidably housed in the syringe outer cylinder 1. Furthermore, a plunger 5 attached to the gasket 7 may be provided.

In addition, as illustrated in FIGS. 15, 16, and 47, the prefilled syringe of the present invention includes the syringe outer cylinder 1, 1a, the gasket 7 slidably housed in the syringe outer cylinder 1, 1a, and medical liquid housed in the syringe outer cylinder. Furthermore, a plunger 5 attached to the gasket 7 may be provided.

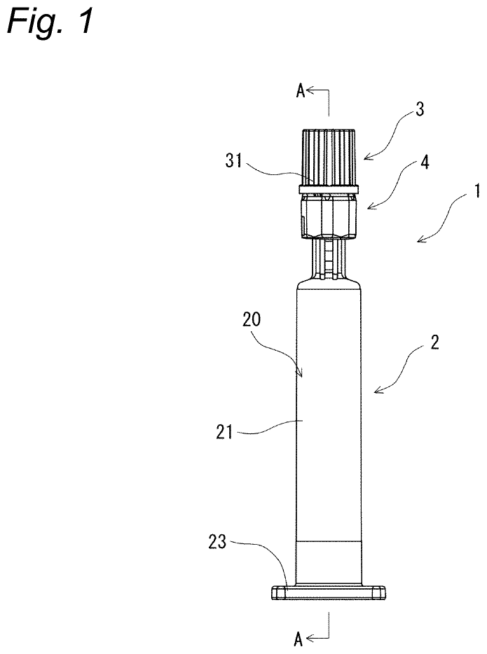

As illustrated in FIGS. 1 to 3, the syringe outer cylinder 1 of the present invention includes the outer cylinder 2 and the cap 3 attachable to and detachable from the outer cylinder 2. Note that, In the drawings, the cap 3 is attached to the outer cylinder 2.

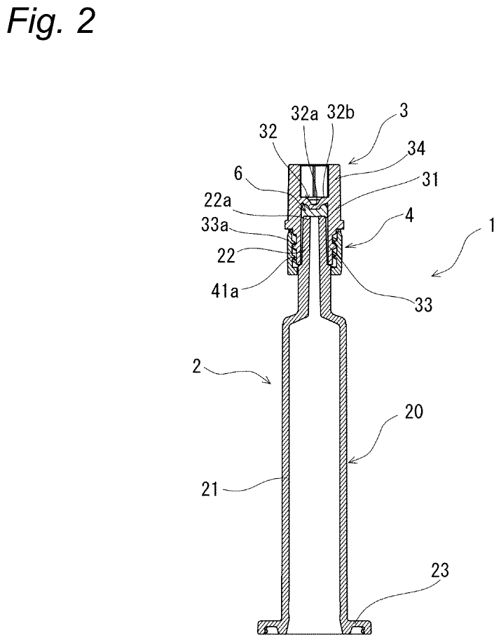

In this embodiment, as illustrated in FIGS. 1 and 2, the outer cylinder 2 includes an outer cylinder body 20 and a lock adapter 4 rotatably attached to the outer cylinder body 20 (specifically, the nozzle part 22). The lock adapter 4 constitutes a locking cylindrical part that covers the nozzle part 22.

The outer cylinder body 20 includes the nozzle part 22 including an opening at a distal end thereof and having a hollow structure, a hollow body part 21, and a flange 23 provided at a proximal end of the hollow body part 21.

The nozzle part 22 can be connected with another medical instrument (an injection needle, a connector, or a three-way stopcock, for example).

The lock adapter 4 includes, on an inner surface thereof, a locking cylindrical part-side screw part (lock adapter-side screw part) 41a that can be screwed with medical instrument (an injection needle, a connector, or a three-way stopcock, for example, that is a rib or screw part formed on a flange) connected with the nozzle part.

The flange 23 has an arc-shaped outer edge that is formed protruding in a direction perpendicular to a central axis of an outer cylinder body part from an entire circumference of the proximal end of the hollow body part 21.

In this embodiment, the hollow body part 21 of the outer cylinder body 20 is formed in a cylindrical shape having a relatively small diameter, and is filled with a small amount (more specifically, 0.5 ml to 2 ml) of a drug solution or the like, and an outer diameter of the hollow body part 21 is 6 to 12 mm. Note that the outer cylinder body 20 is not limited to those having such a small diameter and a capacity.

The nozzle part 22 has a diameter that is smaller than the outer diameter of the hollow body part 21, and that is reduced toward the distal end. The nozzle part 22 includes, at a distal end thereof, a distal opening for discharging the drug solution or the like in the outer cylinder. Furthermore, the nozzle part 22 includes a locking rib that can be locked with the lock adapter 4 at a proximal portion (slightly distal of the distal end of the hollow body part 21). In this embodiment, the locking rib is an annular rib.

Examples of materials for forming the outer cylinder body 20 and the lock adapter 4 include various resins such as polypropylene, polyethylene, polystyrene, polyamide, polycarbonate, polyvinyl chloride, poly-(4-methylpentene-1), acrylic resin, acrylonitrile-butadiene-styrene copolymer, polyester such as polyethylene terephthalate, cyclic polyolefin polymer, and cyclic olefin copolymer. Among these resin materials, resins such as polypropylene, cyclic polyolefin polymer, and cyclic olefin copolymer are easy to mold and have heat resistance, and therefore are preferable. Note that the cyclic olefin polymer and the cyclic olefin copolymer that have such high transparency that a drug solution filled inside the outer cylinder body can be visually checked from an outside, and have heat resistance for withstanding autoclave treatment is particularly preferable as the materials for forming the outer cylinder body 20.

Furthermore, as illustrated in FIGS. 1 to 3, the lock adapter 4 is a cylindrical member, and is rotatably attached to the nozzle part 22 of the outer cylinder body 20. In addition, a lower portion of the lock adapter 4 is held by the locking rib provided on the nozzle part 22. Moreover, the lock adapter 4 includes an adapter-side screw part 41a that can be screwed with the cap-side screw part 33a of the cap 3 described later. In this embodiment, the screw part 41a is formed with two helical ribs.

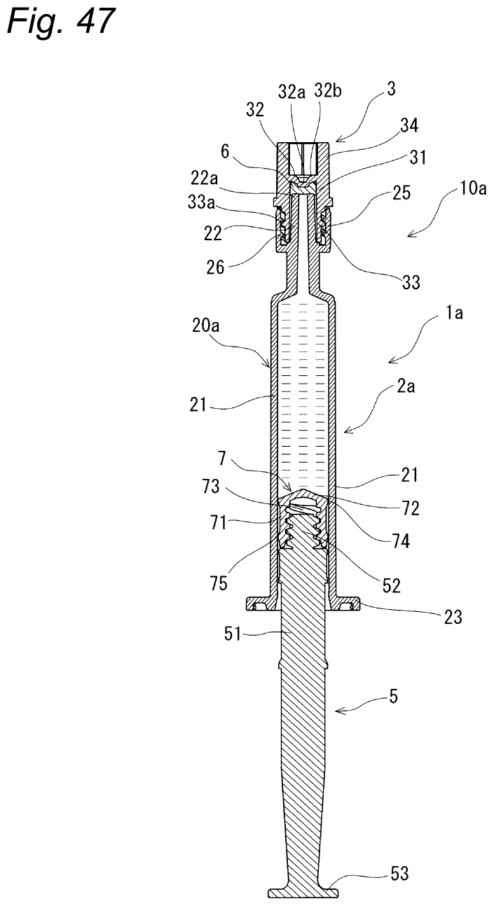

Note that a locking cylindrical part of the outer cylinder 2 is not limited to the lock adapter 4 described above. For example, as in an outer cylinder 2a of a syringe outer cylinder la used for a prefilled syringe 10a of an embodiment illustrated in FIG. 47, the locking cylindrical part may be a collar part 25 integrally formed with the outer cylinder 2a.

The collar part 25 covers the nozzle part 22. The collar part 25 is formed in a cylindrical shape concentrically with the nozzle part 22 so as to surround the nozzle part 22. In addition, a distal end of the collar part 25 is open, and each of an inner diameter and outer diameter of the collar part 25 is substantially the same from a proximal end to a distal end. In addition, the distal portion of the nozzle part 22 protrudes from a distal opening of the collar part 25. Furthermore, on an inner peripheral surface of the collar part 25, there is formed a collar-part side screw part 26 for screwing with the cap-side screw part 33a formed on the nozzle-part housing part 33 of a sealing cap 3.

As a result, the nozzle part 22 of the outer cylinder 2a and the cap 3 are screwed with each other between the inner peripheral surface of the collar part 25 and an outer peripheral surface of a nozzle housing part. In addition, a screw part 26 of the collar part 25 can be screwed with a screw part on a side close to a connector (for example, a needleless connector) provided on an injection needle hub, a blood circuit, or a drug solution administration line. In this embodiment, both the cap-side screw part 33a and the collar-part side screw part 26 are formed with the helical ribs and can be screwed with each other.

The cap 3 is a cylindrical member, and includes a sealing-member housing part 31 that houses an elastic sealing member 6, a closing part 32 positioned at an upper portion of a sealing-member housing part 31, the nozzle-part housing part 33 that extends downward from the sealing-member housing part 31 and houses the nozzle part 22 (specifically, a distal-end-side portion 22a of the nozzle part 22), and the cap-side screw part 33a formed on an outer surface of the nozzle-part housing part 33. In addition, in this embodiment, the cap 3 includes an upper cylindrical part 34 extending upward from the closing part 32. The upper cylindrical part 34 is used as a grip part at a time of detaching the cap 3 from the outer cylinder 2.

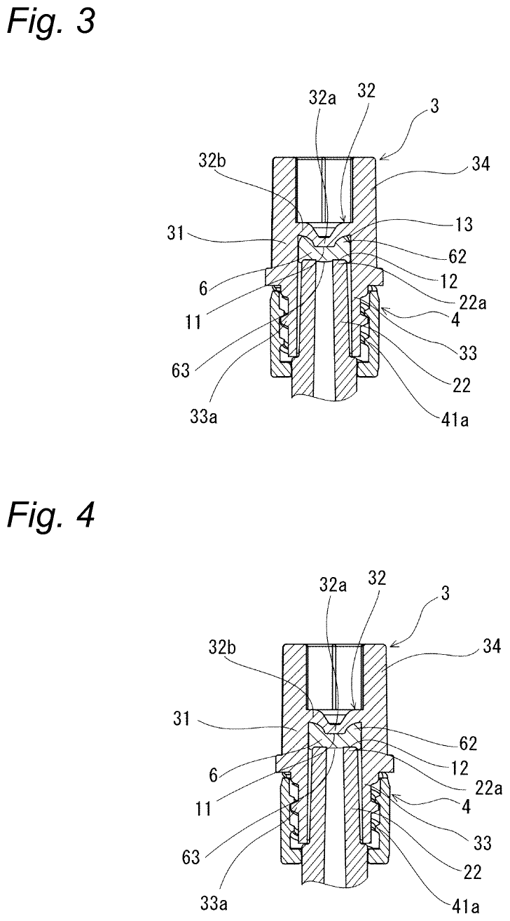

As illustrated in FIGS. 2 and 3, the cap-side screw part 33a of the cap 3 can be screwed with the adapter-side screw part 41a of the lock adapter 4 described above. In this embodiment, the cap-side screw part 33a is formed with two helical ribs.

Examples of materials for forming the cap 3 include various resins such as polypropylene, polyethylene, polystyrene, polyamide, polycarbonate, polyvinyl chloride, poly-(4-methylpentene-1), acrylic resin, acrylonitrile-butadiene-styrene copolymer, polyester such as polyethylene terephthalate, and cyclic polyolefin. Among these resin materials, resins such as polypropylene, and cyclic polyolefin are easy to mold and have heat resistance, and therefore are preferable.

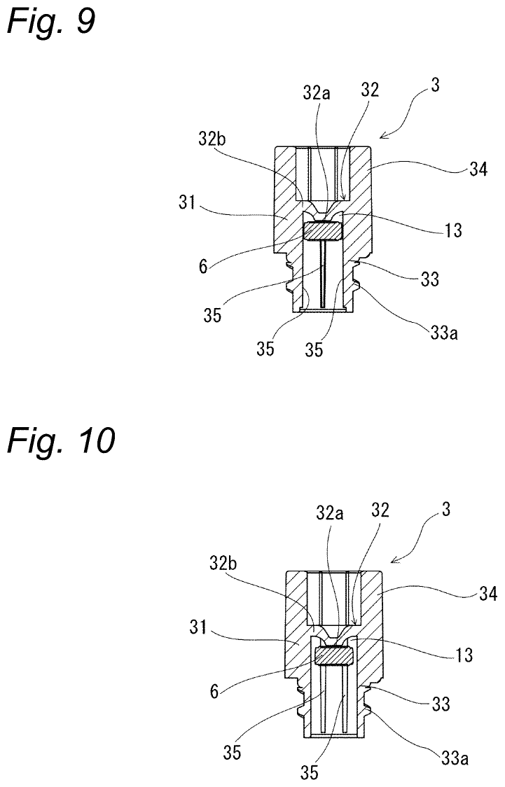

As illustrated in FIG. 9, the cap 3 includes, at least before being attached to the outer cylinder 2 (when not attached), the gap portion 13 formed between the upper portion (upper peripheral portion) of the sealing-member housing part 31 and an upper portion of the elastic sealing member 6. Furthermore, in the syringe outer cylinder 1 of this embodiment, as illustrated in FIG. 3, when attached to the outer cylinder 2, the elastic sealing member 6 is compressed by an inner surface 11 of the cap 3 and a distal end 12 of the nozzle part 22 and elastically deformed. Furthermore, as illustrated in FIG. 3, a swollen portion 62 of the elastic sealing member 6 formed by the elastic deformation is positioned in (enters) the gap portion 13.

Used for the elastic sealing member 6 is an elastic material that is elastically deformable by compression between the inner surface 11 of the cap 3 and the distal end 12 of the nozzle part 22, and is configured to liquid tightly seal the distal opening of the nozzle part 22.

Used for the elastic sealing member 6 is, for example, an elastically deformable material such as natural rubber, synthetic rubber such as isoprene rubber, butadiene rubber, fluororubber, or silicone rubber, or thermoplastic elastomers such as olefin-based elastomer or styrene-based elastomer.

Furthermore, in the syringe outer cylinder 1 of this embodiment, as illustrated in FIGS. 2, 3, 9, and 10, the cap 3 includes a lower-central protruding part 32a provided on the closing part 32 and having a planar lower surface. In addition, an annular part 32b is provided on an upper peripheral portion of the lower-central protruding part 32a. Specifically, an outer surface and inner surface of the lower-central protruding part 32a of the closing part 32 are reduced in diameter toward a lower end of the lower-central protruding part 32a, and the lower end of the lower-central protruding part 32a has a planar lower surface. In other words, a side surface of the lower-central protruding part 32a has a diameter increasing upward (toward the annular part 32b) from the planar lower surface. Furthermore, on a side portion of the lower-central protruding part 32a, there is formed the gap portion 13 having an annular shape and having a tapered inner peripheral surface in which the inner peripheral surface increases in diameter upward.

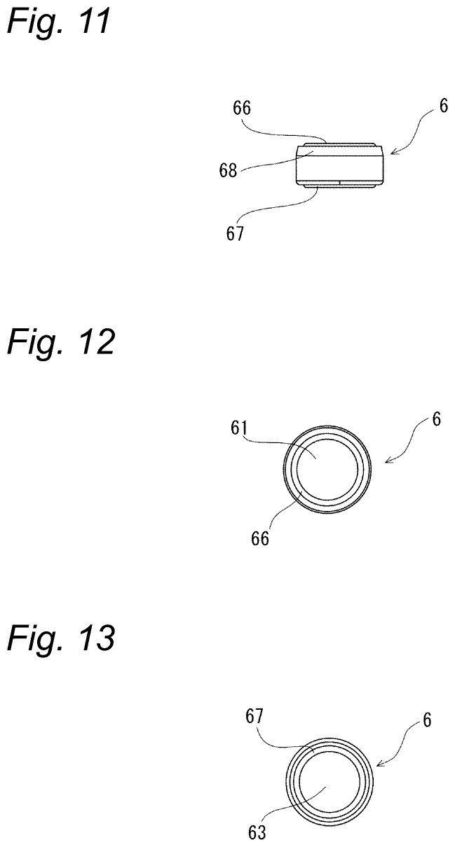

Furthermore, in the syringe outer cylinder 1 of this embodiment, as illustrated in FIGS. 8 to 13, the elastic sealing member 6 has a columnar shape. Specifically, as illustrated in FIGS. 11 to 13, the elastic sealing member 6 of this embodiment is formed in a columnar shape, and includes an upper surface annular rib 66, an upper surface central part (upper-surface planar part) 61 surrounded by the upper surface annular rib 66, a distal-end tapered part 68, a lower surface annular rib 67, and a lower-surface planar part 63 surrounded by the lower surface annular rib 67.

Furthermore, as illustrated in FIGS. 9 and 10, the cap 3 of this embodiment has the gap portion 13 having an annular shape and formed between the upper portion of the sealing-member housing part 31 and the upper portion of the elastic sealing member 6 in a state in which the cap 3 is not attached to the outer cylinder 2.

Specifically, the annular gap portion 13 is a gap portion formed between an annular side surface of the lower-central protruding part 32a, an upper inner-side surface of the nozzle-part housing part 33, and the upper portion of the elastic sealing member 6, and having an annular shape, an outer diameter substantially uniform, an inner diameter gradually decreasing downward, and a width gradually decreasing upward. In addition, the annular gap portion 13 is thicker in a peripheral direction.

In addition, in the cap 3 of this embodiment, as illustrated in FIG. 3, in a state in which the cap 3 is attached to the outer cylinder 2, the cap-side screw part 33a of the cap 3 and the adapter-side screw part 41a of the lock adapter 4 are screwed with each other, and the elastic sealing member 6 liquid tightly seals the distal opening of the nozzle part 22, the upper surface central part (upper-surface planar part) 61 of the elastic sealing member 6 is pressed downward by the planar lower surface of the lower-central protruding part 32a of the closing part 32 of the cap 3, and the lower-surface planar part 63 is pressed by a distal end surface of the nozzle part 22 of the outer cylinder 2.

Therefore, the elastic sealing member 6 is compressed by the inner surface of the cap 3 and the distal end of the nozzle part 22 and elastically deformed. Specifically, the upper surface central part 61 of the elastic sealing member 6 is pressed downward by which a central part is recessed as illustrated in FIG. 3. An upper portion of the elastic sealing member 6 is swollen upward. In other words, the elastic sealing member 6 is deformed in an axial direction. The annular swollen portion 62 extending in the axial direction is formed. The annular swollen portion 62 enters the annular gap portion 13.

In this embodiment, as illustrated in FIG. 3, in a state in which the cap 3 is attached to the outer cylinder 2, the swollen portion (deformed portion) of the elastic sealing member 6 can enter the annular gap portion 13, and thus generation of the repulsive force due to compression of the elastic sealing member 6 is reduced. Therefore, detachment of the cap 3 from the lock adapter 4 due to repulsion caused by the compression of the elastic sealing member 6 is prevented.

In addition, in this embodiment, the upper surface central part 61 of the elastic sealing member 6 is pressed in a circular shape by the planar lower surface of the lower-central protruding part 32a of the closing part 32 of the cap 3, and the lower-surface planar part 63 of the elastic sealing member 6 is pressed in a ring shape by the distal end surface of the nozzle part 22 of the outer cylinder 2.

Note that, in this embodiment, as illustrated in FIG. 3, the annular swollen portion 62 does not fill the entire annular gap portion 13, and a part of the annular gap portion 13, the upper peripheral portion of the annular gap portion in a case of FIG. 3, remains. Moreover, in this embodiment, as illustrated in FIG. 3, a part, which is the lower-surface planar part 63 of the elastic sealing member 6 and is positioned in the distal opening of the nozzle part 22, is slightly swollen and enters the distal opening of the nozzle part 22. Note that, as illustrated in FIG. 4, the cap 3 may have a structure in which, in a state in which the cap 3 is attached to the outer cylinder 2, the annular swollen portion 62 fills the entire annular gap portion 13, by which the annular gap portion 13 substantially disappears.

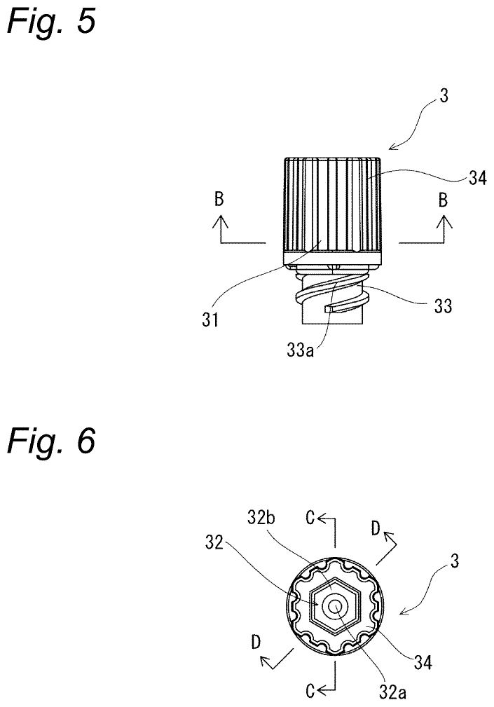

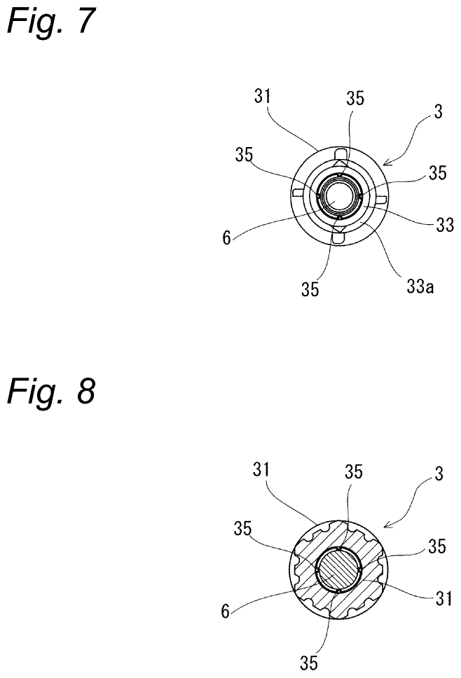

Moreover, in the cap 3 of this embodiment, as illustrated in FIGS. 5 to 10, the cap 3 includes a plurality of ribs 35 extending in a direction from a side of the nozzle-part housing part 33 toward the closing part 32. As illustrated in FIG. 8, a side surface of the elastic sealing member 6 is in contact with and held by the plurality of ribs 35. The elastic sealing member 6 may be pressed by the plurality of ribs 35 to form a recess. Note that, in this embodiment, in a state in which the cap 3 is not attached to the outer cylinder 2, as illustrated in FIGS. 8 and 10, the elastic sealing member 6 has an outer surface separated from an inner surface of the cap 3, except for the plurality of ribs 35.

In addition, in the cap 3 of this embodiment, the plurality of ribs 35 is provided so as to be substantially equiangular with respect to a central axis of the cap. The number of the ribs 35 is preferably about three to six, and in this embodiment, there are four ribs. In addition, the ribs 35 extend in a lower end direction from an upper end part of the inner surface of the cap 3 and terminates at a portion that does not reach the lower end. Moreover, terminal end surfaces of the ribs 35 may be inclined surfaces that are lowered toward a terminal end direction.

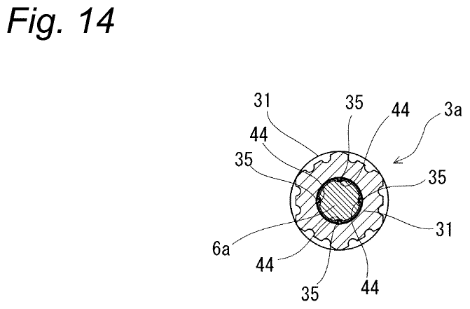

Moreover, similarly to the cap 3a illustrated in FIG. 14, an elastic sealing member 6a may include, on a side surface thereof, a plurality of rib recesses 44 configured to receive distal-end-side portions of the plurality of ribs 35. In this embodiment, in a state in which the cap 3a is not attached to the outer cylinder 2, as illustrated in FIG. 14, the elastic sealing member 6a has an outer surface in proximity to an inner surface of the cap 3a, except for the plurality of ribs 35.

As illustrated in FIGS. 15, 16, and 47, the syringe of the present invention includes the syringe outer cylinder 1, 1a, the gasket 7 slidably housed in the syringe outer cylinder 1, 1a, and the plunger 5 attached to the gasket 7.

In addition, as illustrated in FIGS. 15, 16, and 47, the prefilled syringe 10, 10a of the present invention include the syringe outer cylinder 1, 1a, the gasket 7 slidably housed in the syringe outer cylinder 1, 1a, the medical liquid housed in the syringe outer cylinder, and the plunger 5 attached to the gasket 7.

As the gasket 7, a known gasket is used. The illustrated gasket 7 includes the cylindrical body part 71, a closed distal portion 72, a distal-end-side rib 74 formed on an outer surface of the cylindrical body part 71, a rear-end-side rib 75, and an inner cavity part 73 formed on an inner surface. The gasket includes a screw rib on an inner surface of the inner cavity part 73.

As the plunger 5, a known plunger is used. The illustrated plunger 5 includes a body part 51 having a flange part configured to press a proximal end surface of the gasket 7 on the distal end side, and a distal portion 52 protruding forward from the body part 51 and configured to enter the inner cavity part 73 of the gasket 7. A plunger pressing part 53 is provided on a proximal end of the body part 51. The distal portion 52 is a small-diameter cylindrical part, and includes a screw rib that can be screwed with the screw rib provided on the inner surface of the inner cavity part 73 of the gasket 7.

As the medical liquid, a known medical liquid can be used. Specifically, as the medical liquid, a drug solution, a flash liquid, a contrast agent, or the like is used.

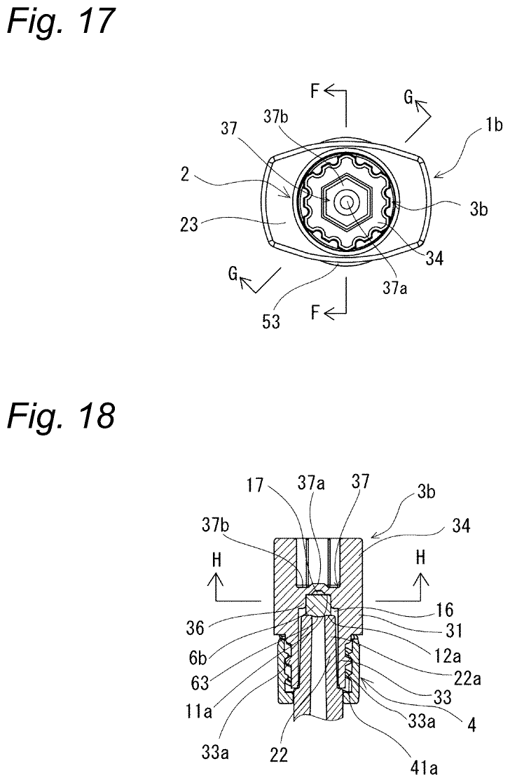

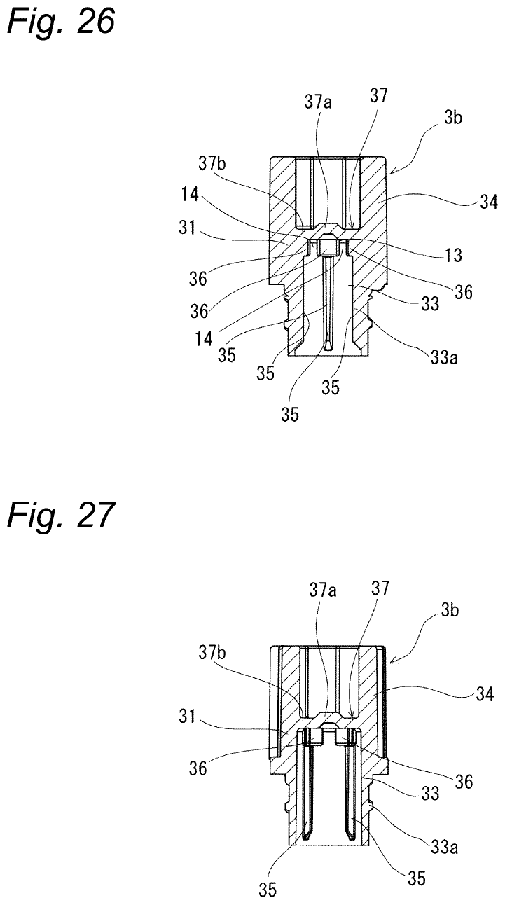

Next, a syringe outer cylinder 1b of the embodiment illustrated in FIGS. 17 to 27 will be described.

In the syringe outer cylinder 1b of this embodiment also, an outer cylinder 2 identical to the outer cylinder 2 in the syringe outer cylinder 1 described above is used.

Furthermore, in the syringe outer cylinder 1b, a cap 3b and a sealing member 6b are used.

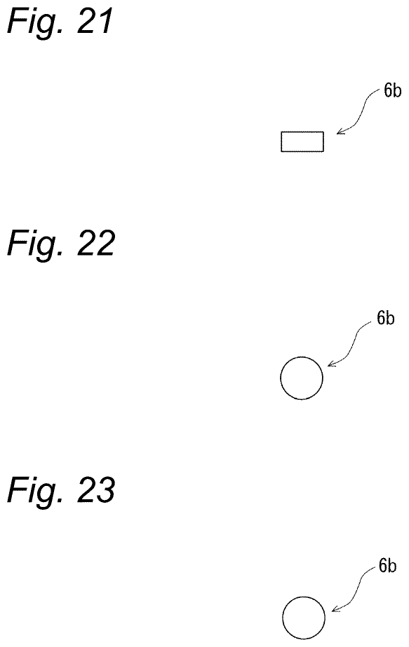

As illustrated in FIGS. 21 to 23, the sealing member 6b is a columnar body having a lower surface in contact with an annular distal end surface having a distal opening of a nozzle part 22. Specifically, the sealing member 6b has a columnar shape extending with substantially the same outer diameter. The sealing member 6b includes an upper surface flat part and a lower surface flat part. The lower surface flat part is in contact with the annular distal end surface having the distal opening of the nozzle part 22.

As illustrated in FIGS. 25 to 27, the cap 3b includes a plurality of inner side-surface protruding parts 36 provided on a sealing-member housing part 31, and a gap portion 14 is a plurality of side gap portions 14 formed between adjacent inner side-surface protruding parts 36. In addition, in the cap 3b of this embodiment, the plurality of inner side-surface protruding parts 36 is provided so as to be substantially equiangular with respect to a central axis of the cap. The number of the inner side-surface protruding parts 36 is preferably about three to six, and in this embodiment, there are four inner side-surface protruding parts 36.

The cap 3b is a cylindrical member, and includes the sealing-member housing part 31 that houses the elastic sealing member 6b, a closing part 37 positioned at an upper portion of the sealing-member housing part 31, a nozzle-part housing part 33 that extends downward from the sealing-member housing part 31 and houses the nozzle part 22 (specifically, a distal-end-side portion of the nozzle part 22), and a cap-side screw part 33a formed on an outer surface of the nozzle-part housing part 33.

The cap-side screw part 33a of the cap 3b can be screwed with an adapter-side screw part 41a of a lock adapter 4 described above. In this embodiment, the cap-side screw part 33a is formed with two helical ribs.

As a material for forming the cap 3b, the material for the cap 3 described above can be suitably used.

Furthermore, as illustrated in FIGS. 18, 19, and 25 to 27, the cap 3b includes an upper-central protruding part 37a provided on the closing part 37. An annular part 37b is provided on a peripheral portion of the upper-central protruding part 37a. An outer surface and inner surface of the upper-central protruding part 37a are reduced in diameter toward an upper end of the upper-central protruding part 37a, and an upper end of the upper-central protruding part 37a has a planar shape.

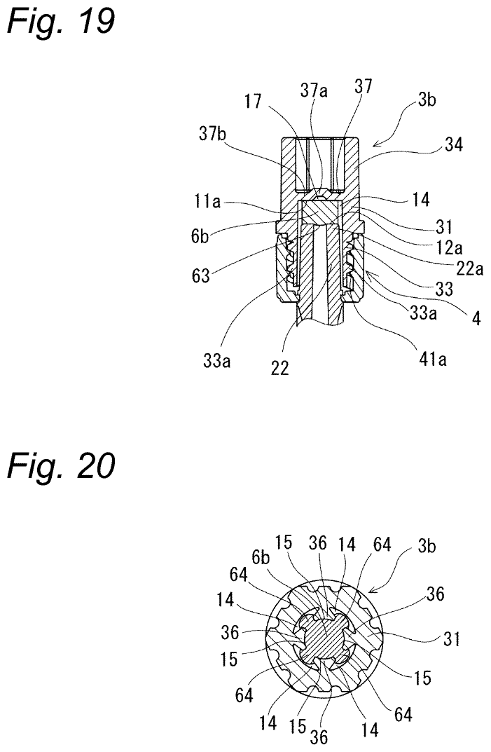

In addition, an annular flat surface 11a is provided on a lower surface of the closing part 37, and the annular flat surface 11a comes into contact with an upper portion of the sealing member 6b as illustrated in FIGS. 18 and 19. In addition, a clearance 17 is formed inside the upper-central protruding part 37a.

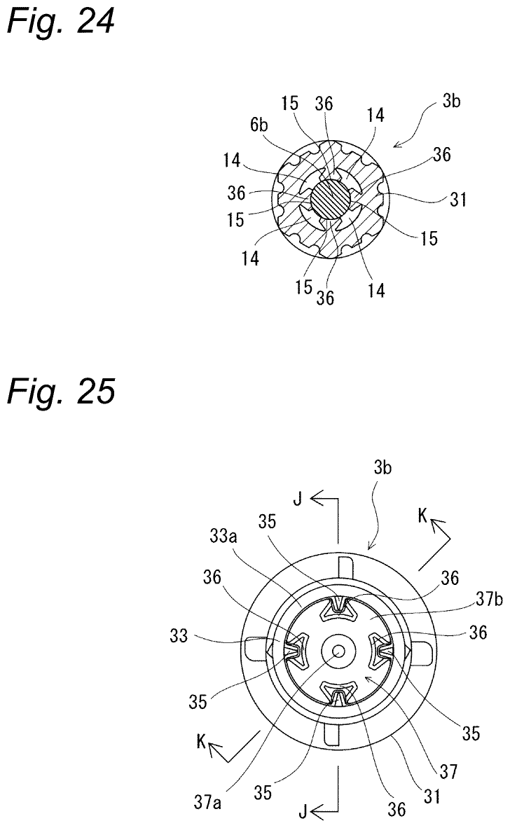

Furthermore, as illustrated in FIG. 24, in a state in which the cap 3b is not attached to the outer cylinder 2, the cap 3b of this embodiment includes the plurality of side gap portions 14 formed between the inner side-surface protruding parts 36 adjacent to a side surface of the elastic sealing member 6b. The plurality of side gap portions 14 is isolated and do not communicate with each other. In addition, the plurality of inner side-surface protruding parts 36 of the cap 3b extends in a direction of the center of the cap 3b, and has a width increasing in a center direction. Moreover, each of the distal end surfaces of the plurality of inner side-surface protruding parts 36 includes an arc-shaped recess extending in an axial direction. Therefore, as illustrated in FIG. 24, each of the distal end surfaces of the plurality of inner side-surface protruding parts 36 faces a side surface of the elastic sealing member 6 and can be favorably brought into contact with the side surface. In this embodiment, as illustrated in FIG. 24, a width of each of the plurality of side gap portions 14 increases in a peripheral direction.

Furthermore, in this embodiment, as illustrated in FIG. 18, a clearance part 16 having an annular shape is formed on a lower portion of the sealing-member housing part 31, which is a lower portion of the plurality of inner side-surface protruding parts 36, and the clearance part 16 having an annular shape communicates with the plurality of side gap portions 14. Note that the plurality of inner side-surface protruding parts 36 is only required not to come into contact with the distal end surface of the nozzle part 22 when attached to the outer cylinder, and lower ends of the plurality of inner side-surface protruding parts 36 may be in proximity to the distal end surface of the nozzle part 22 when attached to the outer cylinder.

In addition, in the cap 3b of this embodiment, as illustrated in FIGS. 18, 19, and 20, in a state in which the cap 3b is attached to the outer cylinder 2, the cap-side screw part 33a of the cap 3b and the adapter-side screw part 41a of the lock adapter 4 are screwed with each other, and the elastic sealing member 6b liquid tightly seals the distal opening of the nozzle part 22, the upper portion of the elastic sealing member 6b is pressed downward by the annular flat surface 11a of the lower surface of the closing part 37 of the cap 3b, and a lower surface of the elastic sealing member 6b is pressed by a distal end 12a of the nozzle part 22 of the outer cylinder 2. In addition, a side-surface portion of the elastic sealing member 6b is in contact with the distal end surface of each of the inner side-surface protruding parts 36 at each of contact portions 15, and is pressed.

Therefore, the elastic sealing member 6b is compressed by an inner surface of the cap 3b and the distal end of the nozzle part 22 and elastically deformed. Specifically, as illustrated in FIG. 20, a side portion of the elastic sealing member 6b is partially swollen, in other words, the elastic sealing member 6b is partially deformed in a circumferential direction (side portion direction, gap portion direction) to form a plurality of swollen portions 64 extending in the circumferential direction (side portion direction, gap portion direction), and each of the swollen portions 64 enters a corresponding gap portion 14.

In this embodiment, as illustrated in FIG. 20, in a state in which the cap 3b is attached to the outer cylinder 2, the swollen portions 64 (deformed portions) of the elastic sealing member 6b can enter the gap portions 14, and thus generation of the repulsive force due to compression of the elastic sealing member 6b is reduced. Therefore, detachment of the cap 3b from the lock adapter 4 due to repulsion caused by the compression of the elastic sealing member 6b is prevented.

In addition, in this embodiment, the upper portion of the elastic sealing member 6b is pressed in a ring shape by the annular flat surface 11a, which is a lower surface of the annular part 37b of the closing part 37, a lower-surface planar part of the elastic sealing member 6b is pressed in a ring shape by the distal end surface of the nozzle part 22 of the outer cylinder 2, and the side-surface portion of the elastic sealing member 6b is pressed by the distal end surfaces of the inner side-surface protruding parts 36.

Note that, in this embodiment, as illustrated in FIG. 20, each of the entering swollen portions 64 does not fill the entire gap portions 14, and a part of the gap portions 14, side portions of the gap portions 14 in a case of FIG. 20, remains. Moreover, in this embodiment, as illustrated in FIGS. 18 and 19, a portion positioned in the distal opening of the nozzle part 22 of the elastic sealing member 6b is slightly swollen and enters the distal opening of the nozzle part 22. Note that the cap 3b may have a structure in which, in a state in which the cap 3b is attached to the outer cylinder 2, each of the swollen portions 64 enters and fills the entire gap portions 14, by which the gap portions 14 substantially disappear.

Moreover, in the cap 3b of this embodiment, as illustrated in FIGS. 25 to 27, the cap 3b includes a plurality of ribs 35 extending in a direction of a lower end of the cap 3b from the lower end of the plurality of inner side-surface protruding parts 36. In addition, in the cap 3b of this embodiment, the plurality of ribs 35 is provided so as to be substantially equiangular with respect to the central axis of the cap. The number of the ribs 35 is preferably about three to six, and in this embodiment, there are four ribs. In addition, in this embodiment, the ribs 35 extend in a lower end direction from an upper end part of the inner surface of the cap 3b and terminates at a portion that does not reach the lower end. Moreover, terminal end surfaces of the ribs 35 are inclined surfaces that are lowered toward a terminal end direction.

Used for the elastic sealing member 6b is an elastic material that is elastically deformable by compression between the inner surface of the cap 3b and the distal end of the nozzle part 22, and is configured to liquid tightly seal the distal opening of the nozzle part 22. As a material for forming the elastic sealing member 6b, the material for the elastic sealing member 6 described above can be suitably used.

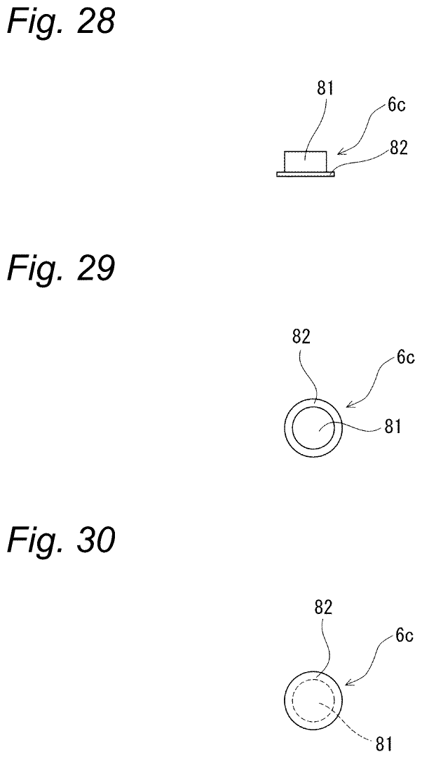

In addition, as the syringe outer cylinder of the present invention, an elastic sealing member 6c illustrated in FIGS. 28 to 30 may be used.

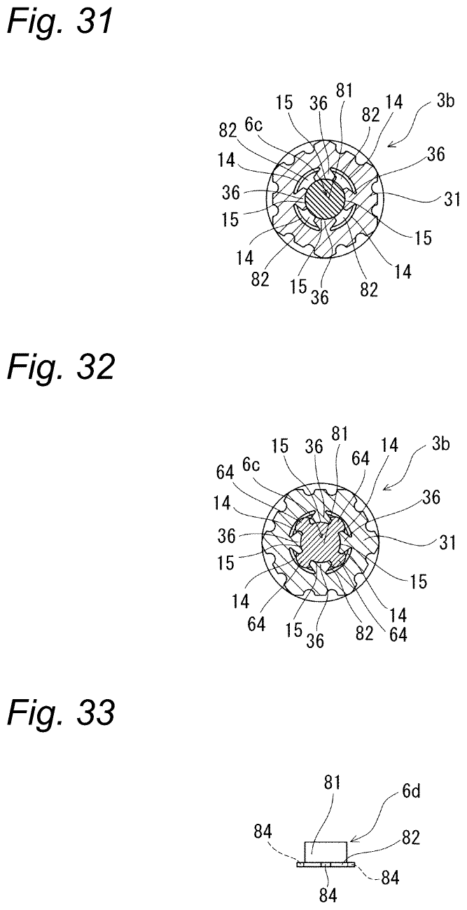

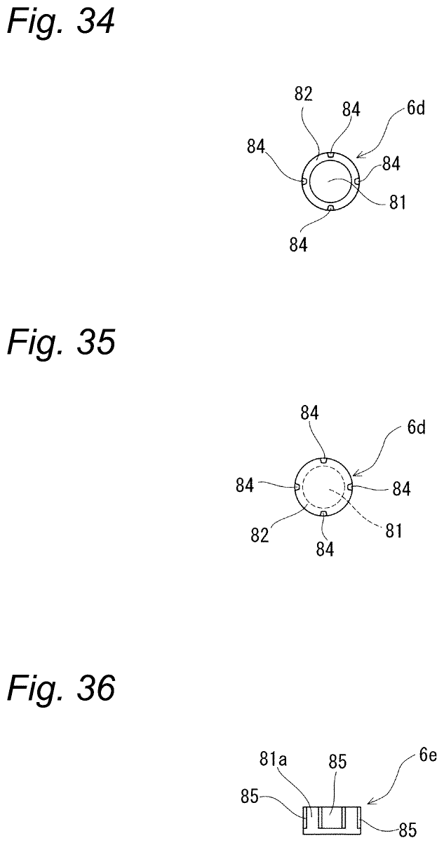

As illustrated in FIGS. 28 to 30, the sealing member 6c is a columnar body having a lower surface in contact with an annular distal end surface having the distal opening of the nozzle part 22, and includes a columnar-shaped body part 81 and an annular flange 82 provided on a lower portion of the columnar-shaped body part 81. As illustrated in FIGS. 31 and 32, the sealing member 6c can be used for the cap 3b illustrated in FIGS. 25 to 27 described above. By providing the annular flange 82, an area in contact with the distal end surface of the nozzle part 22 increases. Moreover, the elastic sealing member may be an elastic sealing member 6d illustrated in FIGS. 33 to 35, the elastic sealing member 6d having, on the annular flange 82, recesses 84 corresponding to the ribs 35 formed on the inner surface of the cap 3b.

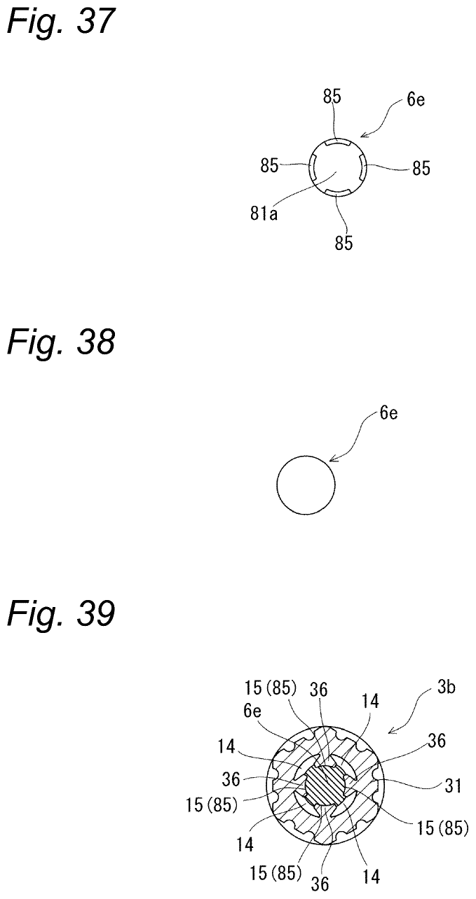

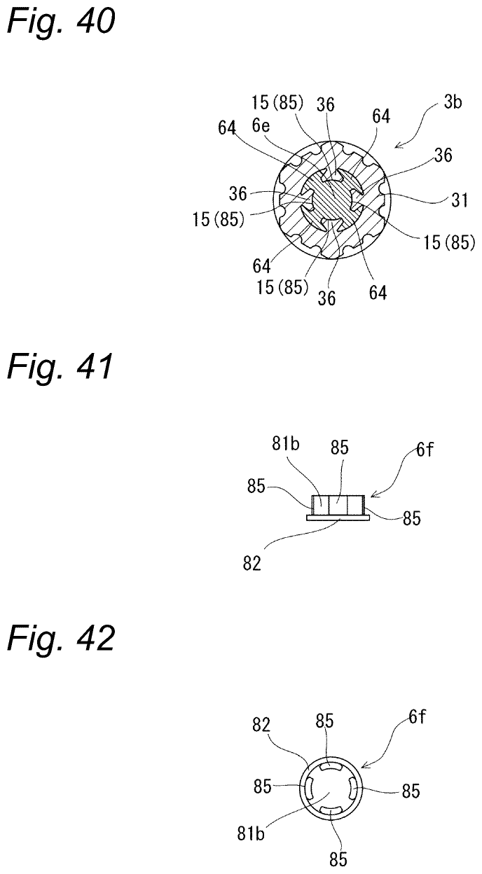

In addition, as the syringe outer cylinder of the present invention, an elastic sealing member 6e illustrated in FIGS. 36 to 38 may be used. As illustrated in FIGS. 39 and 40, the sealing member 6e can be used for the cap 3b illustrated in FIGS. 25 to 27 described above.

As illustrated in FIGS. 36 to 38, the sealing member 6e in this embodiment is a columnar body having a lower surface in contact with an annular distal end surface having the distal opening of the nozzle part 22, and includes a columnar-shaped body part 81a and a plurality of recesses 85 provided on a side portion of the columnar-shaped body part 81a. Specifically, as described above, the cap 3b includes the plurality of inner side-surface protruding parts 36, and the gap portions 14 is a plurality of side gap portions formed between adjacent ones of the inner side-surface protruding parts 36. The elastic sealing member 6e of this embodiment includes a plurality of protruding-part recesses 85 configured to receive distal-end-side portions of the side-surface protruding parts 36 on the side portion of the columnar-shaped body part 81a. Furthermore, as illustrated in FIG. 36, the protruding-part recesses 85 extend in a lower surface direction from an upper surface of the elastic sealing member (columnar-shaped body part 81a) and does not reach the lower surface, and includes a protruding-part recess bottom surface portion (a bottom portion of the protruding-part recesses 85). Therefore, when the cap 3b is attached to the outer cylinder 2, the protruding-part recess bottom surface portion (the bottom portion of the protruding-part recesses 85) is compressed between lower surfaces of the plurality of protruding-part recesses 85 and the distal end surface of the nozzle part 22.

Furthermore, as illustrated in FIG. 39, before the cap 3b is attached to the outer cylinder 2, the distal-end-side portions of the side-surface protruding parts 36 of the cap are housed in the protruding-part recesses 85. In addition, the sealing member 6e positioned at a portion between the protruding-part recesses 85 enters between the side-surface protruding parts 36 that are a part of the gap portions 14. Furthermore, in this embodiment also, when the cap 3b is attached to the outer cylinder 2, as illustrated in FIG. 40, a side portion of the elastic sealing member 6e is partially swollen, in other words, the elastic sealing member 6e is partially deformed in the circumferential direction to form the plurality of swollen portions 64 extending in the circumferential direction, and each of the swollen portions 64 enters the corresponding gap portion 14.

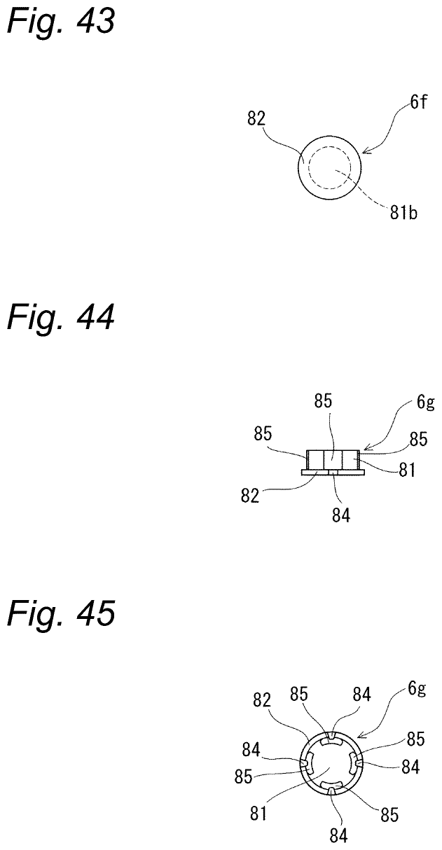

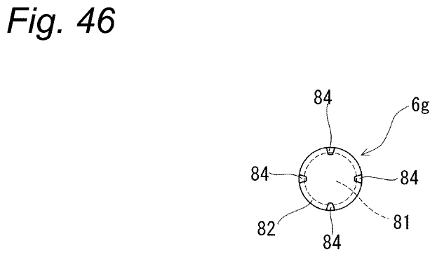

Moreover, the above-described sealing member 6e may include the annular flange 82 provided on a lower portion of a columnar-shaped body part 81b as in a sealing member 6f illustrated in FIGS. 41 to 43. By providing the annular flange 82, an area in contact with the distal end surface of the nozzle part 22 increases. Moreover, the elastic sealing member may be, as an elastic sealing member 6g illustrated in FIGS. 44 to 46, an elastic sealing member having, on the annular flange 82, recesses 84 corresponding to the ribs 35 formed on the inner surface of the cap 3b.

The syringe outer cylinder according to an embodiment of the present disclosure includes an outer cylinder including nozzle part provided at a distal end thereof and a locking cylindrical part covering the nozzle part, a cap attachable to and detachable from the outer cylinder, and an elastic sealing member housed in the cap and being for sealing a distal opening of the nozzle part. Furthermore, the cap is a cylindrical member, and includes a sealing-member housing part that houses the elastic sealing member, a closing part positioned on an upper portion of the sealing-member housing part, a nozzle-part housing part that extends downward from the sealing-member housing part and houses the nozzle part, a cap-side screw part formed on an outer surface of the nozzle- part housing part, and a gap portion formed, at least before being attached to the outer cylinder, between an upper portion of the sealing-member housing part and an upper surface peripheral portion of the elastic sealing member, or between an inner-side portion of the sealing-member housing part and an outer-side portion of the elastic sealing member, the locking cylindrical part-side screw part of the outer cylinder includes, in an inner surface, a locking cylindrical part-side screw part that is configured to be screwed with the cap-side screw part, and the elastic sealing member is compressed by an inner surface of the cap and a distal end of the nozzle part and elastically deformed when the cap is attached to the outer cylinder, and a swollen portion formed by the elastic deformation is positioned in the gap portion.

In the syringe outer cylinder described above, generation of repulsive force of the sealing member can be reduced, because the cap is attached to the nozzle part of the outer cylinder, and the swollen portion (deformed portion) resulting from squeezing enters the gap portion described above even if the sealing member is compressed and the amount of squeezing the sealing member is large. Therefore, there is an extremely low possibility that the cap is detached from the syringe outer cylinder to which the cap is attached.

A syringe outer cylinder according to certain embodiments of the present disclosure is as follows.
(1) A syringe outer cylinder including an outer cylinder including a nozzle part provided on a distal end, and a locking cylindrical part covering the nozzle part, a cap attachable to and detachable from the outer cylinder, and
an elastic sealing member for sealing a distal opening of the nozzle part, the elastic sealing member being housed in the cap, in which
the cap is a cylindrical member, and includes a sealing-member housing part that houses the elastic sealing member, a closing part positioned on an upper portion of the sealing-member housing part, a nozzle-part housing part that extends downward from the sealing-member housing part and houses the nozzle part, a cap-side screw part formed on an outer surface of the nozzle-part housing part, and a gap portion formed, at least before being attached to the outer cylinder, between an upper portion of the sealing-member housing part and an upper surface peripheral portion of the elastic sealing member, or between an inner-side portion of the sealing-member housing part and an outer-side portion of the elastic sealing member,
the locking cylindrical part of the outer cylinder includes, in an inner surface, a locking cylindrical part-side screw part that is configured to be screwed with the cap-side screw part, and
the elastic sealing member is compressed by an inner surface of the cap and a distal end of the nozzle part and elastically deformed when the cap is attached to the outer cylinder, and a swollen portion formed by the elastic deformation is positioned in the gap portion.

The syringe outer cylinder includes an outer cylinder including nozzle part provided at a distal end thereof and a locking cylindrical part covering the nozzle part, a cap attachable to and detachable from the outer cylinder, and an elastic sealing member housed in the cap and being for sealing a distal opening of the nozzle part. Furthermore, the cap is a cylindrical member, and includes a sealing-member housing part that houses the elastic sealing member, a closing part positioned on an upper portion of the sealing-member housing part, a nozzle-part housing part that extends downward from the sealing-member housing part and houses the nozzle part, a cap-side screw part formed on an outer surface of the nozzle-part housing part, and a gap portion formed, at least before being attached to the outer cylinder, between an upper portion of the sealing-member housing part and an upper surface peripheral portion of the elastic sealing member, or between an inner-side portion of the sealing-member housing part and an outer-side portion of the elastic sealing member, the locking cylindrical part of the outer cylinder includes, in an inner surface, a locking cylindrical part-side screw part that is able to be screwed with the cap-side screw part, and the elastic sealing member is compressed by an inner surface of the cap and a distal end of the nozzle part and elastically deformed when the cap is attached to the outer cylinder, and a swollen portion formed by the elastic deformation is positioned in the gap portion.

In the syringe outer cylinder described above, generation of repulsive force of the sealing member can be reduced, because the cap is attached to the nozzle part of the outer cylinder, and the swollen portion (deformed portion) resulting from squeezing enters the gap portion described above even if the sealing member is compressed and the amount of squeezing the sealing member is large. Therefore, there is an extremely low possibility that the cap is detached from the syringe outer cylinder to which the cap is attached.

In addition, the embodiments described may be configured as follows.
(2) The syringe outer cylinder according to (1), in which the locking cylindrical part is a lock adapter rotatably attached to the nozzle part.
(3) The syringe outer cylinder according to (1), in which the locking cylindrical part is a collar part integrally formed with the outer cylinder.
(4) The syringe outer cylinder according to any one of (1) to (3), in which the elastic sealing member has a columnar shape.
(5) The syringe outer cylinder according to any one of (1) to (3), in which the elastic sealing member includes a columnar-shaped body part and an annular flange provided on a lower portion of the columnar-shaped body part.
(6) The syringe outer cylinder according to any one of (1) to (5), in which the cap includes a plurality of ribs extending in a direction from a side of the nozzle-part housing part toward the closing part, and a side surface of the elastic sealing member is pressed by the plurality of ribs.
(7) The syringe outer cylinder according to any one of (1) to (6), in which the cap includes a plurality of ribs extending in a direction from a side of the nozzle-part housing part toward the closing part, and the elastic sealing member includes a plurality of rib recesses configured to receive a distal-end-side portion of the ribs.
(8) The syringe outer cylinder according to any one of (1) to (7), in which the elastic sealing member is a columnar body having a lower surface in contact with an annular distal end surface having a distal opening of the nozzle part.
(9) The syringe outer cylinder according to any one of (1) to (8), in which the closing part includes a lower-central protruding part having a planar lower surface, and the elastic sealing member has an upper surface central part pressed downward by the planar lower surface of the lower-central protruding part.
(10) The syringe outer cylinder according to (9), in which the gap portion is an annular gap portion formed between a side surface of the lower-central protruding part of the closing part, an inner-side-surface upper portion of the cap, and the upper surface peripheral portion of the elastic sealing member.
(11) The syringe outer cylinder according to (9) or (10), in which the gap portion is an annular gap portion formed between a side surface of the lower-central protruding part of the closing part, an inner-side-surface upper portion of the cap, and the upper surface peripheral portion of the elastic sealing member, and, when the cap is attached to the outer cylinder, an upper annular swollen portion formed by elastic deformation of the elastic sealing member and extending upward in an axial direction enters the gap portion.
(12) The syringe outer cylinder according to any one of (1) to (8), in which the cap includes a plurality of inner side-surface protruding parts provided on the sealing-member housing part, and the gap portion is a plurality of side gap portions formed between adjacent ones of the inner side-surface protruding parts.
(13) The syringe outer cylinder according to any one of (1) to (8) or (12), in which the cap includes a plurality of inner side-surface protruding parts provided on the sealing-member housing part, the gap portion is a plurality of side gap portions formed between adjacent ones of the inner side-surface protruding parts, and when the cap is attached to the outer cylinder, a plurality of side swollen portions formed by elastic deformation of the elastic sealing member enters the side gap portions.

The syringe of the present invention is as follows.
(14) A syringe including
the syringe outer cylinder according to any one of (1) to (13), and
a gasket slidably housed in the syringe outer cylinder.

The prefilled syringe of the present invention is as follows.
(15) A prefilled syringe including
the syringe outer cylinder according to any one of (1) to (13), the syringe outer cylinder having the cap attached to the outer cylinder,
a gasket slidably housed in the syringe outer cylinder, and
medical liquid housed in the syringe outer cylinder.

1. A syringe outer cylinder comprising:
an outer cylinder comprising:
a nozzle part located at a distal end of the outer cylinder, and
a locking cylindrical part covering the nozzle part;
a cap attachable to and detachable from the outer cylinder; and
an elastic sealing member for sealing a distal opening of the nozzle part, the elastic sealing member being housed in the cap; wherein:
the cap is cylindrical and comprises:
a sealing-member housing part that houses the elastic sealing member,
a closing part positioned on an upper portion of the sealing-member housing part,
a nozzle-part housing part that extends downward from the sealing-member housing part and houses the nozzle part,
a cap-side screw part formed on an outer surface of the nozzle-part housing part, and
a gap portion formed, at least before the cap is attached to the outer cylinder, between an upper portion of the sealing-member housing part and an upper surface peripheral portion of the elastic sealing member, or between an inner-side portion of the sealing-member housing part and an outer-side portion of the elastic sealing member;
the locking cylindrical part of the outer cylinder comprises, at an inner surface, a locking cylindrical part-side screw part that is configured to be screwed with the cap-side screw part; and
when the cap is attached to the outer cylinder, the elastic sealing member is compressed by an inner surface of the cap and a distal end of the nozzle part and elastically deformed, and a swollen portion formed by the elastic deformation is positioned in the gap portion.

2. The syringe outer cylinder according to claim 1, wherein the locking cylindrical part is a lock adapter rotatably attached to the nozzle part.

3. The syringe outer cylinder according to claim 1, wherein the locking cylindrical part is a collar part integrally formed with the outer cylinder.

4. The syringe outer cylinder according to claim 1, wherein the elastic sealing member has a columnar shape.

5. The syringe outer cylinder according to claim 1, wherein the elastic sealing member comprises a columnar-shaped body part, and an annular flange located at a lower portion of the columnar-shaped body part.

6. The syringe outer cylinder according to claim 1, wherein:
the cap comprises a plurality of ribs extending in a direction from a side of the nozzle-part housing part toward the closing part; and
a side surface of the elastic sealing member is pressed by the plurality of ribs.

7. The syringe outer cylinder according to claim 1, wherein:
the cap comprises a plurality of ribs extending in a direction from a side of the nozzle-part housing part toward the closing part; and
the elastic sealing member includes a plurality of rib recesses configured to receive a distal-end-side portion of the ribs.

8. The syringe outer cylinder according to claim 1, wherein the elastic sealing member is a columnar body having a lower surface in contact with an annular distal end surface of the nozzle part, the annular distal end surface having a distal opening.

9. The syringe outer cylinder according to claim 1, wherein:
the closing part comprises a lower-central protruding part having a planar lower surface; and
the elastic sealing member has an upper surface central part pressed downward by the planar lower surface of the lower-central protruding part.

10. The syringe outer cylinder according to claim 9, wherein:
the gap portion is an annular gap portion formed between a side surface of the lower-central protruding part of the closing part, an inner-side-surface upper portion of the cap, and the upper surface peripheral portion of the elastic sealing member.

11. The syringe outer cylinder according to claim 9, wherein:
the gap portion is an annular gap portion formed between a side surface of the lower-central protruding part of the closing part, an inner-side-surface upper portion of the cap, and the upper surface peripheral portion of the elastic sealing member; and
when the cap is attached to the outer cylinder, an upper annular swollen portion formed by elastic deformation of the elastic sealing member and extending upward in an axial direction enters the gap portion.

12. The syringe outer cylinder according to claim 1, wherein:
the cap comprises a plurality of inner side-surface protruding parts located at the sealing-member housing part; and
the gap portion comprises a plurality of side gap portions formed between adjacent ones of the inner side-surface protruding parts.

13. The syringe outer cylinder according to claim 1, wherein:
the cap comprises a plurality of inner side-surface protruding parts located at the sealing-member housing part;
the gap portion comprises a plurality of side gap portions formed between adjacent ones of the inner side-surface protruding parts; and
when the cap is attached to the outer cylinder, a plurality of side swollen portions formed by elastic deformation of the elastic sealing member enter the side gap portions.

14. A syringe comprising:
the syringe outer cylinder according to claim 1 and
a gasket slidably housed in the syringe outer cylinder.

15. A prefilled syringe comprising:
the syringe outer cylinder according to claim 1, the syringe outer cylinder having the cap attached to the outer cylinder;
a gasket slidably housed in the syringe outer cylinder; and
medical liquid housed in the syringe outer cylinder.

16. A syringe outer cylinder comprising:
an outer cylinder comprising:
a nozzle located at a distal end of the outer cylinder, and
a lock adapter covering the nozzle;
a cap attachable to and detachable from the outer cylinder; and
an elastic sealing member for sealing a distal opening of the nozzle, the elastic sealing member being housed in the cap; wherein:
the cap is cylindrical and comprises:
a sealing-member housing part that houses the elastic sealing member,
a closing part positioned on an upper portion of the sealing-member housing part,
a nozzle housing part that extends downward from the sealing-member housing part and houses the nozzle,
a cap-side screw part formed on an outer surface of the nozzle housing part, and
a gap formed, at least before the cap is attached to the outer cylinder, between an upper portion of the sealing-member housing part and an upper surface peripheral portion of the elastic sealing member, or between an inner-side portion of the sealing-member housing part and an outer-side portion of the elastic sealing member;
the lock adapter of the outer cylinder comprises, at an inner surface, a lock adapter side screw part that is configured to be screwed with the cap-side screw part; and
when the cap is attached to the outer cylinder, the elastic sealing member is compressed by an inner surface of the cap and a distal end of the nozzle and elastically deformed, and a swollen portion formed by the elastic deformation is positioned in the gap portion.

17. A method of forming a syringe outer cylinder, the method comprising:
providing an outer cylinder comprising:
a nozzle part located at a distal end of the outer cylinder, and
a locking cylindrical part covering the nozzle part and comprising, at an inner surface, a locking cylindrical part-side screw part;
providing a cap attachable to and detachable from the outer cylinder, and an elastic sealing member housed in the cap for sealing a distal opening of the nozzle part, wherein the cap is cylindrical and comprises:
a sealing-member housing part that houses the elastic sealing member,
a closing part positioned on an upper portion of the sealing-member housing part,
a nozzle-part housing part that extends downward from the sealing-member housing part and houses the nozzle part,
a cap-side screw part formed on an outer surface of the nozzle-part housing part and configured to be screwed with the locking cylindrical part-side screw part, and
a gap portion formed, at least before the cap is attached to the outer cylinder, between an upper portion of the sealing-member housing part and an upper surface peripheral portion of the elastic sealing member, or between an inner-side portion of the sealing-member housing part and an outer-side portion of the elastic sealing member; and
attaching the cap to the outer cylinder so that the elastic sealing member is compressed by an inner surface of the cap and a distal end of the nozzle part and elastically deformed, and a swollen portion formed by the elastic deformation is positioned in the gap portion.
